(19) United States
(12) Patent Application Publication
(10) Pub. No.: US 2026/0179759 A1
(43) Pub. Date: Jun. 25, 2026

(54) SYSTEMS AND METHODS FOR PREDICTING DIAGNOSIS OF CHRONIC BACK PAIN OF A PATIENT AND A CORRESPONDING TREATMENT BASED ON ONE OR MORE IMAGES OR INFOGRAPHICS OF THE PATIENT'S BACK

(71) Applicant: Spinal Diagnostic LLC, Orlando, FL (US)

(72) Inventor: Ara Deukmedjian, Orlando, FL (US)

(73) Assignee: Spinal Diagnostic LLC, Orlando, FL (US)

(21) Appl. No.: 19/457,893

(22) Filed: Jan. 23, 2026

Related U.S. Application Data
(63) Continuation of application No. 19/287,684, filed on Jul. 31, 2025.
(60) Provisional application No. 63/737,777, filed on Dec. 22, 2024.

(51) Int. Cl. G16H 30/40; G06N 20/00; G06T 7/00; G16H 10/60; G16H 30/20; G16H 50/20; G16H 50/30; G16H 70/00

(52) U.S. Cl. CPC G16H 30/40; G06N 20/00; G06T 7/0012; G16H 10/60; G16H 30/20; G16H 50/20; G16H 50/30; G16H 70/00; G06T 2207/20081; G06T 2207/20084; G06T 2207/30012; G06T 2207/30204

(57) ABSTRACT

Methods and systems are provided for diagnosing chronic spinal pain that include receiving and storing an image of a patient's back or neck. The image includes a first set of visual indicia corresponding to anatomical landmarks of the patient's back or neck as well as a second set of visual indica corresponding to locus of pain experienced by the patient. The image is processed to generate feature data representing spatial location and pain characteristics of point(s) corresponding to locus of pain experienced by the patient. The feature data is input to an expert system that generates output data representing a predicted diagnosis of chronic pain of the patient and optional output data representing a confidence level associated with the predicted diagnosis as indicated by the feature data input. A lookup table provides a treatment corresponding to the output data.

### CROSS-REFERENCE TO RELATED APPLICATIONS

This application is a continuation of U.S. Ser. No. 19/287,684, filed Jul. 31, 2025, which claims priority from U.S. Provisional Appl. No. 63/737,777, filed on Dec. 22, 2024, which are herein incorporated by reference in their entireties.

### BACKGROUND

### 1. Field

The present disclosure relates to systems and methods that enable diagnosis of medical conditions, such as the cause of chronic pain of the back, based on patient information transmitted to a central computer system.

### 2. State of the Art

The spine, from the neck to the lower back, is affected by many factors. For example, vertebrae and discs can degenerate with age. Some common spine problems include:
Arthritic conditions, such as ankylosing spondylitis;
Curvature of the spine (scoliosis and kyphosis);
Neuromuscular diseases, such a amyotrophic lateral sclerosis;
Nerve injuries, such as spinal stenosis, sciatica, and pinched nerves;
Spinal cord injuries, including spinal fractures, bulging or herniated discs and paralysis;
Spine tumors and cancer; and
Infections, such as meningitis and osteomyelitis.

Some of these spine problems can cause chronic pain of the spine which can last from many months to years, and sometimes even decades (if left untreated). Chronic pain of the spine can limit the body's mobility and make it challenging for the affected individual to get through daily tasks and activities. It can also cause significant psychological and emotional trauma.

Typically, a patient experiencing chronic pain visits one or more physicians or medical treatment centers to have his or her condition diagnosed. Such visit(s) often require considerable time and expense for the patient to travel to and from the physician's office or medical treatment center. Furthermore, for instances where the patient is experiencing chronic pain of the spine, the cause of such chronic spinal pain can be difficult to properly diagnose via available local medical care, which commonly results in misdiagnosis of the cause of the chronic spinal pain and treatment that does not properly address the cause of the patient's chronic spinal pain.

### SUMMARY

Methods and systems are provided for diagnosing chronic spinal pain of a patient, which involve receiving and storing at least one image or infographic of the patient's back or neck (or portion thereof). The at least one image or infographic of the patient's back or neck (or portion thereof) includes a first set of visual indicia corresponding to anatomical landmarks of the patient's spine as well as a second set of visual indica corresponding to locus of pain experienced by the patient and possibly representing one or more characteristics of the pain at the locus. Optionally, the patient can provide answers or responses to a questionnaire or diagnostic prompts where one or more of the answers or responses include information that characterizes the pain experienced by the patient. For example, the patient can provide responses to diagnostic prompts that inquire about ipsilateral pain (on the same side of the cervical spine as the tender point) when the patient's neck rotates in the lateral direction toward a tender point, which can be used for diagnosis of facet joint injury of the cervical spine as described herein. The at least one image or infographic and optionally one or more of the answers or responses to the questionnaire or diagnostic prompts as provided by the patient can be processed to generate feature data representing spatial location and associated pain characteristics of one or more points corresponding to locus of pain experienced by the patient. The feature data is input to an expert system that generates first output data representing a predicted diagnosis of chronic spinal pain of the patient and optional second output data representing a confidence level associated with the predicted diagnosis as indicated by the feature data input. A lookup table or database can be accessed to determine treatment data representing a treatment corresponding to the first output data and the optional second output data. A medical expert can evaluate the first output data, the optional second output data and the treatment data for confirmation of the diagnosis of chronic spinal pain of the patient and appropriate treatment. When the predicted diagnosis and corresponding treatment are confirmed, the first output data, the optional second output data and the treatment data can be used to provide a diagnosis of chronic spinal pain of the patient and recommended treatment for the diagnosis.

In embodiments, at least part of the operations of the method or system can be performed by a cloud computing system, and the at least one image or infographic can be generated by a user device remote from the cloud computing system and communicated to the cloud computing system from the user device through data communication over the Internet.

In embodiments that diagnose chronic pain of the lower back (e.g., lumbar spine), the first set of visual indicia can include a line AF1 down the center of the lumbar spine of the patient, a line AF2 across the bottom of the lumbar spine that crosses the tailbone/sacrum of the spine of the patient, and a line AF3 across the midline of the lumbar spine between the L2 and L3 vertebrae of the lumbar spine of the patient.

In embodiments that diagnose chronic pain of the upper back (e.g., thoracic spine) of the patient, the first set of visual indicia can include a line AF4 down the center of the thoracic spine of the patient, a line AF5 across the bottom of the thoracic spine that is aligned with the bottom of the rib cage of the patient, and a line AF6 across the top of the thoracic spine that is aligned with the top of the opposed shoulder blades of the patient.

In embodiments that diagnose chronic pain of the neck (e.g., cervical spine) of the patient, the first set of visual indicia can include a line AF7 down the center of the cervical spine of the patient, a line AF8 across the bottom of the cervical spine that is aligned with the top of the opposed shoulder blades of the patient, and a line AF9 across the top of the cervical spine that is aligned with the base of the skull of the patient.

In embodiments, the first set of visual indicia can be marked on the skin of the back or neck of the patient using marker(s), pen(s), tape, or stickers.

In embodiments, the first set of visual indicia can be marked on the skin of the back or neck of the patient by the patient or family member or friend or other person at home or at other premises remote from a doctor's office or treatment center.

In embodiments, the second set of visual indicia can include a circular or other shaped marking at one or more locations corresponding to locus of pain experienced by the patient.

In embodiments, visual distinction between the visual indicia of the first and second sets can use varying color or patterns or other visual properties.

In embodiments, the visual indicia of the second set can correspond to different pain characteristics (e.g., pain from applied pressure v. pain independent of applied pressure, and/or dull pain or sharp pain when pressure is applied).

In embodiments, visual distinction between the visual indicia of the different pain characteristics of the second set can use varying color or patterns or other visual properties.

In embodiments, the second set of visual indicia can be marked on the skin of the back or neck of the patient using marker(s) or pen(s) or stickers.

In embodiments, the second set of visual indicia can be marked on the skin of the back or neck of the patient by the patient or family member or friend or other person at home or at other premises remote from the doctor's office or treatment center.

In embodiments, the expert system can include a rule-based system. In embodiments, the expert system can include a machine learning system trained from training data comprising feature data representing location of chronic spinal pain experienced by a patient and associated label data representing diagnosis of chronic spinal pain and optionally an associated confidence level.

In another aspect, a kit can be provided that includes at least one first applique or decal configured to mark the back or neck of the patient with a first set of visual indicia corresponding to anatomical landmarks of the patient's back or neck, at least one second applique or decal configured to mark the back or neck of the patient with a second set of visual indicia corresponding to locus of pain experienced by the patient, and a printed article of manufacture that embodies or refers to written instructions for marking the back or neck of the patient with i) the first set of visual indicia provided by the at least first applique or decal and ii) the second set of visual indicia provided by the at least one second applique or decal.

In embodiments, the at least one first applique or decal can be formed by a flexible substrate having a first surface coated with a skin-adhering adhesive and a second surface disposed opposite the first surface, wherein the second surface provides visual indicia for marking an underlying anatomical landmark.

In embodiments, the at least one first applique or decal can include a peel-away protective film that covers the first surface.

In embodiments, the at least one first applique or decal can be packaged in a roll.

In embodiments, the at least one second applique or decal can be formed by a flexible substrate having a first surface coated with a skin-adhering adhesive and a second surface disposed opposite the first surface, wherein the second surface provides visual indicia for marking locus of pain experienced by the patient.

In embodiments, the at least one second applique or decal can include a peel-away protective film that covers the first surface.

In embodiments, the at least one second applique or decal can be packaged in a sheet.

In embodiments, the at least one second applique or decal can include a plurality of second appliques or decals with respective second surfaces that employ color, patterns or other visual properties to mark the locus of pain experienced by the patient as well as different characteristics of the pain. The visual indicia may include at least two distinct sets of indicia that can visually represent the different characteristics of the pain.

In embodiments, the printed article of manufacture can include a part that encodes a URL that references written instructions for marking the back or neck of the patient with i) the first set of visual indicia provided by the at least one first applique or decal kit and ii) the second set of visual indicia provided by at least one second applique or decal.

Other aspects are described and claimed herein.

### BRIEF DESCRIPTION OF THE DRAWINGS

FIG. 1 is a schematic diagram of an exemplary system that predicts a diagnosis of chronic spinal pain of a patient and a corresponding treatment based on one or more images or infographics of the patient's back or neck.

FIGS. 2A to 2C, collectively, is a flowchart illustrating an exemplary workflow carried out by the system of FIG. 1, which involves prediction of a diagnosis of chronic spinal pain of a patient and a corresponding treatment based on one or more images or infographics of the patient's back or neck.

FIG. 3 is a schematic illustration of the lower back of an example patient with the skin of the lower back marked with a first set of visual indicia (lines AF1, AF2, AF3) corresponding to anatomical landmarks of the lumbar spine of the patient.

FIG. 4 is a schematic illustration of the lower back of an example patient together with circles and corresponding labels for vertebral joints or bones and vertebral discs of the lumbar spine that represent the locus of pain commonly experienced by patients together with a first set of visual indicia (lines AF1, AF2, AF3) corresponding to anatomical landmarks of the lumbar spine of FIG. 3.

FIG. 5A is a schematic illustration of the lower back of an example patient that includes a first set of visual indicia (lines AF1, AF2, AF3) marked on the skin of the lower back that correspond to anatomical landmarks of the lower back of the patient together with a second set of visual indicia marked on the skin of the lower back of the patient, which includes a single pain point (PP) which is located at a locus of lower back pain experienced by the patient.

FIG. 5B is another schematic illustration of the lower back of another example patient that includes a first set of visual indicia (lines AF1, AF2, AF3) marked on the skin of the lower back that correspond to anatomical landmarks of the lower back of the patient together with a second set of visual indicia marked on the skin of the lower back of the patient, which includes a single tender point (TP) which is located at a locus of lower back pain experienced by the patient.

FIG. 6A illustrates example visual indicia and pain point feature data extracted from an image or infographic of a patient's back by the workflow of FIGS. 2A to 2C.

FIG. 6B illustrates example visual indicia and tender point feature data extracted from an image or infographic of a patient's back by the workflow of FIGS. 2A to 2C.

FIG. 7 is a schematic diagram that illustrates example operations of the workflow of the present disclosure.

FIG. 8 is a flow chart that illustrates example operations for constructing a machine-learning system, which can be used as the expert system in FIG. 7.

FIG. 9 is a schematic illustration of the upper back of an example patient together with circles and corresponding labels for clusters of vertebral joints and clusters of vertebral discs of the thoracic spine that represent the locus of pain commonly experienced by patients together with a first set of visual indicia (lines AF4, AF5, AF6) corresponding to anatomical landmarks of the thoracic spine.

FIG. 10 is a schematic illustration of the neck of an example patient together with circles and corresponding labels for clusters of vertebral joints and clusters of vertebral discs of the cervical spine that represent the locus of pain commonly experienced by patients together with a first set of visual indicia (lines AF7, AF8, AF9) corresponding to anatomical landmarks of the cervical spine.

FIG. 11A is a perspective view of an exemplary applique or decal that can be configured to mark the back or neck of the patient with a first set of visual indicia corresponding to anatomical landmarks of the patient's back or neck;

FIG. 11B is a schematic cross-sectional view of the exemplary applique or decal of FIG. 11A

FIG. 12A is a top view of exemplary appliques or decals that can be configured to mark the back or neck of the patient with a second set of visual indicia corresponding to locus of pain experienced by the patient;

FIG. 12B is a schematic cross-sectional view of the exemplary appliques or decals of FIG. 12A;

FIG. 13 is a schematic view of an exemplary printed article of manufacture that embodies or refers to written instructions for marking the back or neck of the patient with a first set of visual indicia (corresponding to the anatomical landmarks) provided by the at least applique or decal of a kit and a second set of visual indicia (corresponding to the locus of pain) provided by at least one other applique or decal of the kit.

FIG. 14 illustrates an example computer device that can be configured to implement various embodiments of the methods and processes as discussed in the present application.

### DETAILED DESCRIPTION OF THE PREFERRED EMBODIMENTS

As described herein, an “infographic” is a digital image depicting a schematic plan view of a patient's back.

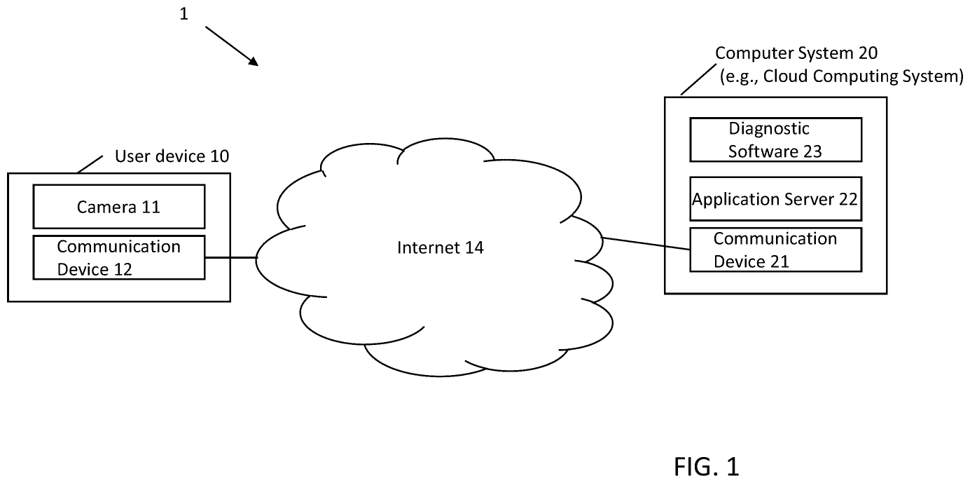

FIG. 1 illustrates components of an exemplary system 1 according to the present disclosure. The system 1 includes two parts, a user device 10 and computer system 20, which are operably coupled to one another by data communication over the Internet 14. The user device 10, which can be a mobile phone, tablet, laptop, pc or other suitable computing device, includes a camera 11 and a communication device 12 that supports data communication over the Internet 14. The computer system 20, which can be a cloud computing system or on-premises computing system, includes a communication device 21 that supports data communication over the Internet 14, an application server 22 and diagnostic software 23. The application server 22 is software that executes on the computer system 20 and hosts the diagnostic software 23 through a communication protocol involving data communication of the Internet 14, including communication with the user device 10. The diagnostic software 23 is a software application that executes on the computer system 20 to carry out tasks configured to predict a diagnosis of chronic spinal pain of a patient and a corresponding treatment based on one or more images or infographics of a patient's back or neck. Such image(s) or infographic(s) includes a first set of visual indicia corresponding to anatomical landmarks of the patient's back or neck as well as a second set of visual indica corresponding to a locus of spinal pain experienced by the patient. The second set of visual indica can also provide one or more characteristics of the pain at the locus.

The user device 10 can be configured to perform the following tasks:
acquire or generate one or images or infographics of the patient's back or neck that includes the first set of visual indicia corresponding to anatomical landmarks of the patient's back or neck as well as the second set of visual indica corresponding to the locus of spinal pain experienced by the patient; the second set of visual indica can also provide one or more characteristics of the pain at the locus; and
communicate or upload the one or more images or infographics of the patient's back or neck to the computer system 20.

The diagnostic software 23 can be configured to perform the following tasks:
receive the one or more images or infographics of the patient's back or neck that is communicated from the user device 10 and store the one or more images or infographics for analysis/processing;
optionally collect answers or responses to a questionnaire or diagnostic prompts where one or more of the answers or responses include information that characterizes the pain experienced by the patient. For example, the patient can provide responses to diagnostic prompts that inquire about ipsilateral pain (on the same side of the cervical spine as the tender point) when the patient's neck rotates in the lateral direction toward a tender point, which can be used for diagnosis of facet joint injury of the cervical spine as described herein;
analyze/process the one or more images or infographics an and optionally one or more answers or responses to the questionnaire or diagnostic prompts to generate feature data representing spatial location and associated pain characteristics of one or more points corresponding to locus of pain experienced by the patient;
input the feature data to an expert system that outputs i) first output data representing a predicted diagnosis of chronic spinal pain of the patient as indicated by the feature data input and optionally ii) second output data representing a confidence level associated with the predicted diagnosis of i);
access a lookup table or database to determine treatment data representing a treatment corresponding to the first output data (predicted diagnosis) and optional second output data (confidence level) output from the expert system;
store the first output data (predicted diagnosis) and optional second output data (confidence level) output from the expert system and the corresponding treatment data for evaluation by a medical expert to confirm the diagnosis of chronic spinal pain of the patient and appropriate treatment; and
when the predicted diagnosis and corresponding treatment are confirmed, use the data representing the predicted diagnosis and corresponding treatment to provide a diagnosis of chronic spinal pain of the patient and recommended treatment for the diagnosis.

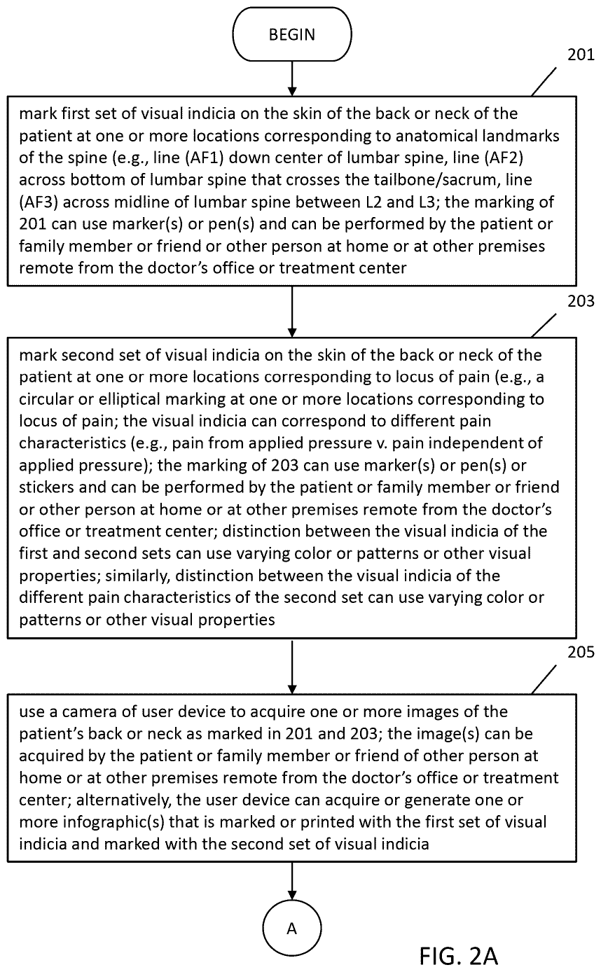

FIG. 2 illustrates an exemplary workflow carried out by the system 1 of FIG. 1 according to the present disclosure.

In 201, a first set of visual indicia is marked on the skin of the back or neck of the patient at one or more locations corresponding to anatomical landmarks.

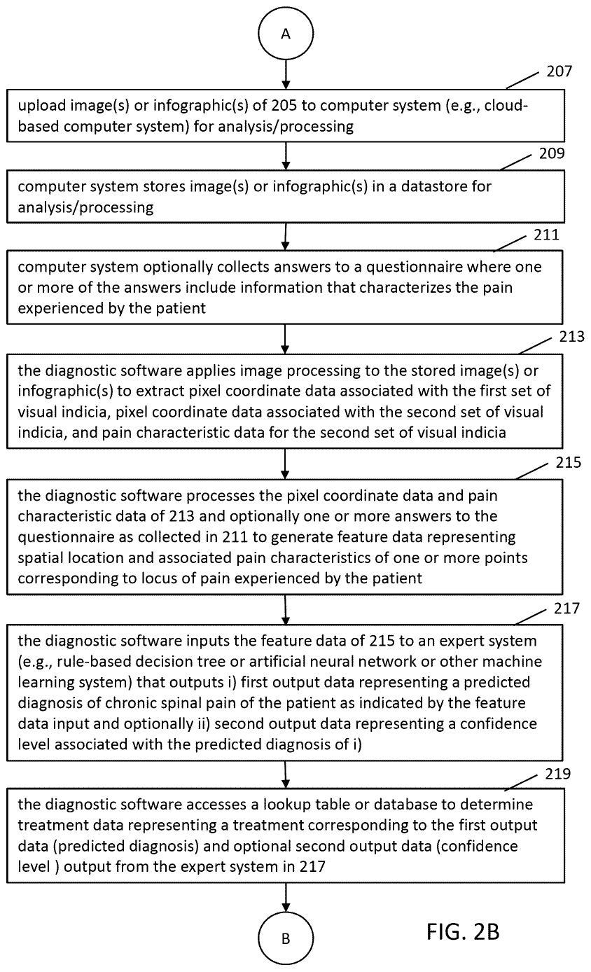

For example, for the case of diagnosing chronic pain of the lower back (e.g., lumbar spine) of the patient, the first set of visual indicia can include a line AF1 down the center of lumbar spine of the patient, a line AF2 across bottom of the spine that crosses the tailbone/sacrum of the spine of the patient, and a line AF3 across the midline of the lumbar spine between the L2 and L3 vertebrae of the lumbar spine of the patient as shown in FIG. 3. The marking of 201 can use marker(s), pen(s), tape, or stickers, and can be performed by the patient or family member or friend or other person at home or at other premises remote from a doctor's office or treatment center.

In another example, for the case of diagnosing chronic pain of the upper back (e.g., thoracic spine) of the patient, the first set of visual indicia can include a line AF4 down the center of the thoracic spine of the patient, a line AF5 across the bottom of the thoracic spine that is aligned with the bottom of the rib cage of the patient, and a line AF6 across the top of the thoracic spine that is aligned with the top of the opposed shoulder blades of the patient.

In yet another example, for the case of diagnosing chronic pain of the neck (e.g., cervical spine) of the patient, the first set of visual indicia can include a line AF7 down the center of the cervical spine of the patient, a line AF8 across the bottom of the cervical spine that is aligned with the top of the opposed shoulder blades of the patient, and a line AF9 across the top of the cervical spine that is aligned with the base of the skull of the patient.

In 203, a second set of visual indicia is marked on the skin of the back or neck of the patient at one or more points or locations corresponding to locus of pain experienced by the patient. For example, the second set of visual indicia can include a circular or other shaped marking at one or more locations corresponding to locus of pain experienced by the patient. In embodiments, the visual indicia of the second set can correspond to different pain characteristics. For example, the visual indicia of the second set can represent a pain point (PP) or a tender point (TP). A pain point corresponds to a location of pain experienced by the patient independent of applied pressure. A tender point corresponds to a location of pain experienced by the patient that results from applied pressure. The visual indicia of the second set can represent other characteristics of the pain experienced by the patient, such as sharp pain or dull pain. Different diagnosis and consequent recommended treatment can arise from the same locus of pain for the different pain characteristics. The marking of 203 can use marker(s) or pen(s) or stickers and can be performed by the patient or family member or friend or other person at home or at other premises remote from the doctor's office or treatment center. The distinction between the visual indicia of the first and second sets can use varying color or patterns or other visual properties. Similarly, distinction between the visual indicia of the different pain characteristics of the second set can use varying color or patterns or other visual properties.

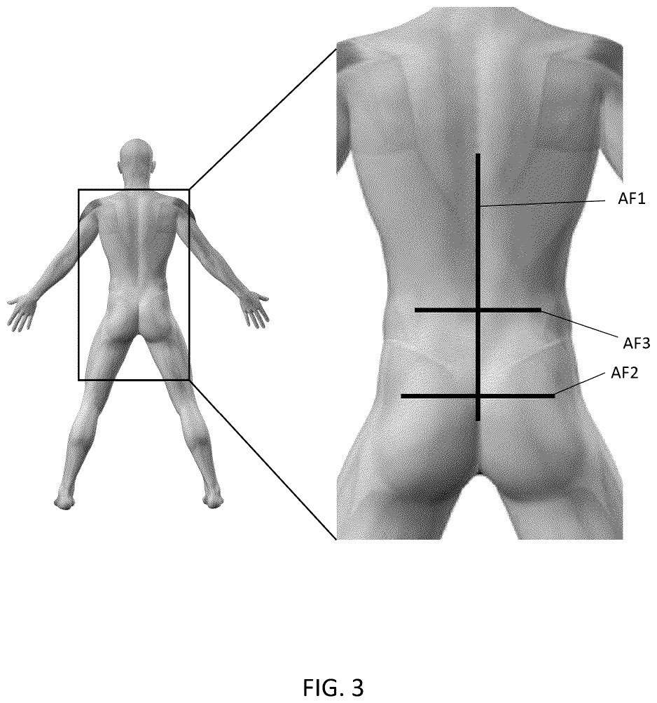

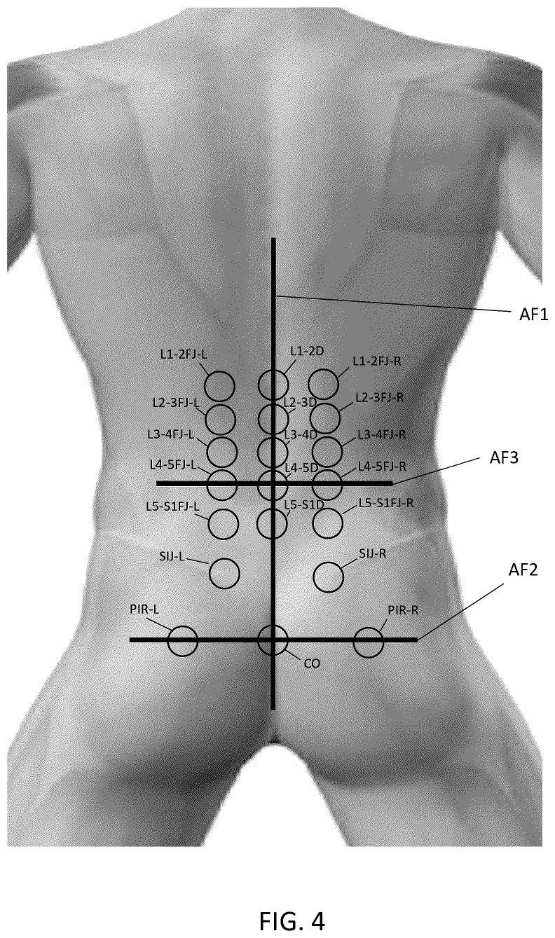

FIG. 5A illustrates the lower back of an example patient that includes a first set of visual indicia (lines AF1, AF2, AF3) marked on the skin of the lower back that correspond to anatomical landmarks of the lower back of the patient together with a second set of visual indicia marked on the skin of the lower back of the patient, which includes a single pain point (PP) which is located at a locus of lower back pain experienced by the patient. The pain point PP corresponds to a location of pain experienced by the patient independent of applied pressure.

FIG. 5B illustrates the lower back of another example patient that includes a first set of visual indicia (lines AF1, AF2, AF3) marked on the skin of the lower back that correspond to anatomical landmarks of the lower back of the patient together with a second set of visual indicia marked on the skin of the lower back of the patient, which includes a single tender point (TP) which is located at a locus of lower back pain experienced by the patient. The tender point TP corresponds to a location of pain experienced by the patient that results from applied pressure.

In 205, the camera of user device 10 is configured to acquire one or more images of the patient's back or neck as marked in 201 and 203. The camera can be operated by the patient (e.g., via a self-timer) or by a family member or friend of other person at home or at other premises remote from the doctor's office or treatment center.

Alternatively, the user device 10 can acquire or generate one or more infographic(s) that is marked or printed with the first set of visual indicia and marked with the second set of visual indicia as described above.

In 207, the user device 10 is configured to upload the image(s) or infographic(s) of 205 to the computer system (e.g., cloud-based computer system) 20 for analysis/processing.

In 209, the computer system 20 stores the image(s) or infographic(s) in a datastore for analysis/processing.

In 211, the computer system 20 optionally collects or responses to a questionnaire or diagnostic prompts where one or more of the answers or responses include information that characterizes the pain experienced by the patient. For example, the patient can provide responses to diagnostic prompts that inquire about ipsilateral pain (on the same side of the cervical spine as the tender point) when the patient's neck rotates in the lateral direction toward a tender point, which can be used for diagnosis of facet joint injury of the cervical spine as described herein.

In 213, the diagnostic software 23 applies image processing to the stored image(s) or infographic(s) to extract pixel coordinate data associated with the first set of visual indicia, pixel coordinate data associated with the second set of visual indicia, and pain characteristic data for the second set of visual indicia.

In 215, the diagnostic software 23 processes the pixel coordinate data and pain characteristic data of 213 and optionally one or more answers or responses to the questionnaire or diagnostic prompts as collected in 211 to generate feature data representing spatial location and associated pain characteristics of one or more points (e.g., such as pain point(s) or tender point(s)) corresponding to locus of pain experienced by the patient.

In 217, the diagnostic software inputs the feature data of 215 to an expert system (e.g., rule-based decision tree or artificial neural network or other machine learning system) that outputs i) first output data representing a predicted diagnosis of chronic spinal pain of the patient as indicated by the feature data input and optionally ii) second output data representing a confidence level associated with the predicted diagnosis of i).

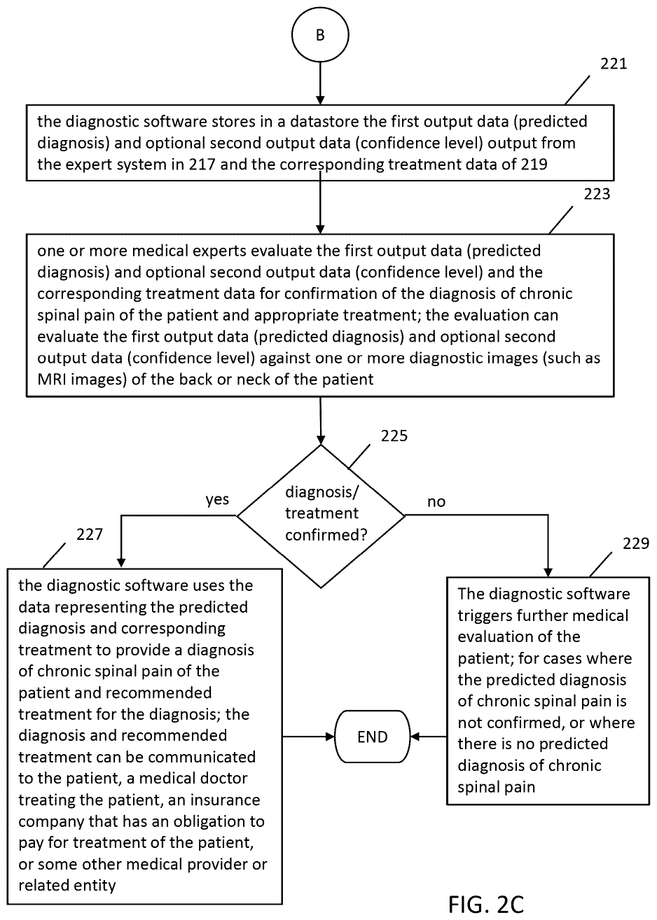

In embodiments, the predicted diagnosis of chronic spinal pain of the patient can refer to one or more vertebral joints or bones or vertebral discs or associated musculature of the lumbar spine of the patient. FIG. 4 illustrates vertebral joints and vertebral discs and associated musculature of the lumbar spine of an example patient that can be referenced by the predicted diagnosis of the first output data of 217, which includes the following:
L1-L2 facet joint of the left side of the lumbar spine, labeled “L1-2FJ-L”;
L1-L2 facet joint of the right side of the lumbar spine, labeled “L1-2FJ-R”;
L2-L3 facet joint of the left side of the lumbar spine, labeled “L2-3FJ-L”;
L2-L3 facet joint of the right side of the lumbar spine, labeled “L2-3FJ-R”;
L3-L4 facet joint of the left side of the lumbar spine, labeled “L3-4FJ-L”;
L3-L4 facet joint of the right side of the lumbar spine, labeled “L3-4FJ-R”;
L4-L5 facet joint of the left side of the lumbar spine, labeled “L4-5FJ-L”;
L4-L5 facet joint of the right side of the lumbar spine, labeled “L4-5FJ-R”;
L5-S1 facet joint of the left side of the lumbar spine, labeled “L5-S1FJ-L”;
L5-S1 facet joint of the right side of the lumbar spine, labeled “L5-S1FJ-R”;
sacroiliac joint of the left side of the lumbar spine, labeled “SIJ-L”;
sacroiliac joint of the right side of the lumbar spine, labeled “SIJ-R”;
piriformis muscle adjacent the left side of the lumbar spine, labeled “PIR-L”;
piriformis muscle adjacent the right side of the lumbar spine, labeled “PIR-R”;
L1-L2 vertebral disc of the lumbar spine, labeled “L1-2D”;
L2-L3 vertebral disc of the lumbar spine, labeled “L2-3D”;
L3-L4 vertebral disc of the lumbar spine, labeled “L3-4D”;
L4-L5 vertebral disc of the lumbar spine, labeled “L4-5D”; and
L5-S1 vertebral disc of the lumbar spine, labeled “L5-S1D”.

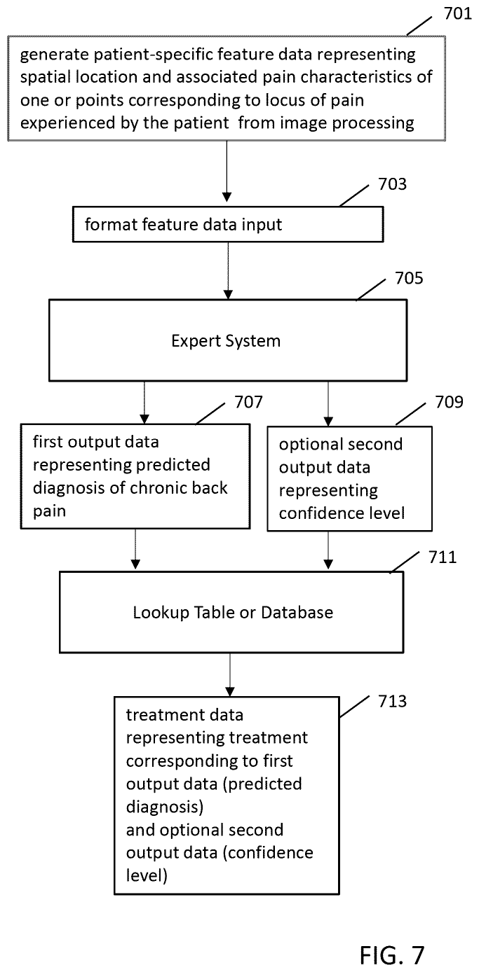

In other embodiments, the predicted diagnosis of chronic spinal pain of the patient can refer to one or more vertebral joints or bones or vertebral discs of the thoracic spine of the patient. FIG. 9 illustrates clusters of vertebral joints and clusters of vertebral discs of the thoracic spine of an example patient that can be referenced by the predicted diagnosis of the first output data of 217, which includes the following:
first cluster of facet joints (which covers the facet joints located between T1 to T4) of the left side of the thoracic spine, labeled “TFC1-L”;
first cluster of facet joints (which covers the facet joints located between T1 to T4) of the right side of the thoracic spine, labeled “TFC1-R”;
second cluster of facet joints (which covers the facet joints located between T4 to T7) of the left side of the thoracic spine, labeled “TFC2-L”;
second cluster of facet joints (which covers the facet joints located between T4 to T7) of the right side of the thoracic spine, labeled “TFC2-R”;
third cluster of facet joints (which covers the facet joints located between T7 to T10) of the left side of the thoracic spine, labeled “TFC3-L”;
third cluster of facet joints (which covers the facet joints located between T7 to T10) of the right side of the thoracic spine, labeled “TFC3-R”;
fourth cluster of facet joints (which covers the facet joints located between T10 to L1) of the left side of the thoracic spine, labeled “TFC4-L”;
fourth cluster of facet joints (which covers the facet joints located between T10 to L1) of the right side of the thoracic spine, labeled “TFC4-R”;
first cluster of vertebral discs (which covers the T1-T2, T2-3, T3-T4 discs) of the thoracic spine, labeled “TDC-1”;
second cluster of vertebral discs (which covers the T4-T5, T5-6, T6-T7 discs) of the thoracic spine, labeled “TDC-2”;
third cluster of vertebral discs (which covers the T7-T8, T8-9, T9-T10 discs) of the thoracic spine, labeled “TDC-3”; and
fourth cluster of vertebral discs (which covers the T10-T11, T11-12, T12-L1 discs) of the thoracic spine, labeled “TDC-4”.

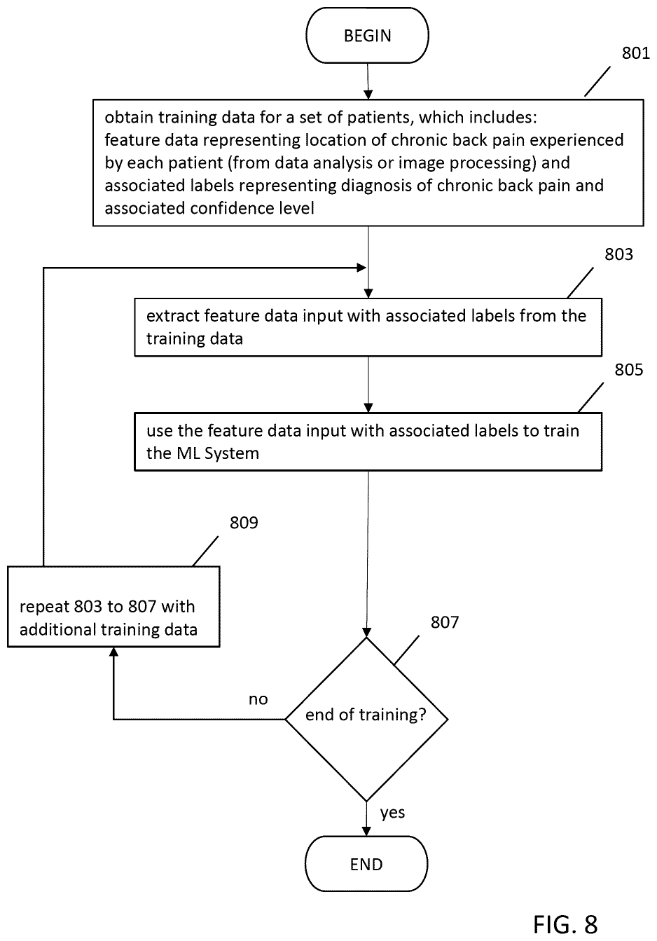

In embodiments, the predicted diagnosis of chronic spinal pain of the patient can refer to one or more vertebral joints or bones or vertebral discs of the cervical spine of the patient. FIG. 10 illustrates clusters of vertebral joints and clusters of vertebral discs of the cervical spine of an example patient that can be referenced by the predicted diagnosis of the first output data of 217, which includes the following:
first cluster of facet joints (which covers the facet joints located between C1 to C5) of the left side of the cervical spine, labeled “CFC1-L”;
first cluster of facet joints (which covers the facet joints located between C1 to C5) of the right side of the cervical spine, labeled “CFC1-R”;
second cluster of facet joints (which covers the facet joints located between C5 to T1) of the left side of the cervical spine, labeled “CFC2-L”;
second cluster of facet joints (which covers the facet joints located between C5 to T1) of the right side of the cervical spine, labeled “CFC2-R”;
first cluster of vertebral discs (which covers the C2-C3, C3-C4, C4-C5 discs) of the cervical spine, labeled “TDC-3”; and
second cluster of vertebral discs (which covers the C5-C6, C6-C7, C7-T1 discs) of the cervical spine, labeled “TDC-4”.

In 219, the diagnostic software 23 accesses a lookup table or database to determine treatment data representing a treatment corresponding to the first output data (predicted diagnosis) and optional second output data (confidence level) output from the expert system in 217.

By way of a first example, the predicted diagnosis of the expert system (the first output data of 217) can refer to chronic pain associated with a disc of the lumbar spine of the patient (which is associated with a pain point (PP) identified at a spatial location corresponding to a disc and identifying persistent pain and absent of tenderness). In this case, a disc herniation, annular tear, annulitis (inflammation) or discogenic pain may be diagnosed. The diagnosis can be further specified or confirmed by analysis of medical images (e.g., MRI images) of the disc of the lumbar spine. The treatment for a diagnosed disk injury or problem would then be advised; such disc repair treatment may include a minimally-invasive treatment, including, but not limited to, a laser disc repair.

By way of second example, the predicted diagnosis of the expert system (the first output data of 217) can refer to chronic pain associated with a facet joint of the lumbar spine of the patient (which is associated with a tender point (TP) identified at a spatial location corresponding to a facet joint and identifying pain and associated tenderness when palpated). In this case, a facet joint injury can be diagnosed, such as resulting from arthritis, arthropathy, hypertrophy, injury, facet adjacent segment disease, or inflammation of the facet joint. The diagnosis can be further specified or confirmed by analysis of medical images (e.g., MRI images) of the facet joint of the lumbar spine. The treatment for the facet joint injury or issue may include, but not be limited to, denervation of the sensory nerves at the indicated facet joint, such as the medial branch of the dorsal ramus. Such denervation may be accomplished via minimally invasive percutaneous cauterization of the sensory nerves extending adjacent the facet joint.

By way of a third example, the predicted diagnosis of the expert system (the first output data of 217) can refer to chronic pain associated with the buttock of the patient, which is associated with a tender point (TP) identified at a spatial location corresponding to the piriformis muscle and identifying tenderness when palpated. In this case, piriformis syndrome may be diagnosed. A treatment for piriformis syndrome would then be advised. Such treatment may include a minimally-invasive percutaneous treatment, including, but not limited to, releasing the piriformis from the greater trochanter of the femur.

By way of a fourth example, the predicted diagnosis of the expert system (the first output data of 217) can refer to chronic pain associated with a disc cluster of the thoracic or cervical spine of the patient (which is associated with a pain point (PP) identified at a spatial location corresponding to a disc cluster and identifying persistent pain and absent of tenderness). In this case, a disc herniation, annular tear, annulitis (inflammation) or discogenic pain may be diagnosed. The diagnosis can be further specified and confirmed for a particular disc of the disc cluster by analysis of medical images (e.g., MRI images) of the disc cluster of the thoracic or cervical spine. The treatment for a diagnosed disk injury or problem would then be advised; such disc repair treatment may include a minimally-invasive treatment, including, but not limited to, a laser disc repair. Alternatively, the PP may be associated with a specific disc, rather than a cluster.

By way of a fifth example, the predicted diagnosis of the expert system (the first output data of 217) can refer to chronic pain associated with a facet joint cluster of the thoracic spine of the patient (which is associated with a tender point (TP) identified at a spatial location corresponding to a facet joint cluster of the thoracic spine and identifying pain and associated tenderness when palpated). In this case, a facet joint injury can be diagnosed, such as resulting from arthritis, arthropathy, hypertrophy, injury, pain, facet adjacent segment disease, or inflammation of the facet joint. The diagnosis can be further specified or confirmed for a particular facet joint of the facet joint cluster by analysis of medical images (e.g., MRI images) of the facet joint cluster of the thoracic spine. The treatment for the facet joint injury or issue may include, but not be limited to, denervation of the sensory nerves at the indicated facet joint, such as the medial branch of the dorsal ramus. Such denervation may be accomplished via minimally invasive percutaneous cauterization of the sensory nerves extending adjacent the facet joint. Alternatively, the TP may be associated with a specific facet joint along the thoracic spine, rather than a cluster of facet joints along the thoracic spine.

By way of a sixth example, the predicted diagnosis of the expert system (the first output data of 217) can refer to chronic pain associated with a facet joint cluster of the cervical spine of the patient (which is associated with a tender point (TP) identified at a spatial location corresponding to a facet joint cluster of the cervical spine and identifying pain and associated tenderness when palpated). The predicted diagnosis can also require that the patient experience ipsilateral pain (on the same side of the cervical spine as the tender point) when the patient's neck rotates in the lateral direction toward the tender point. In this case, a facet joint injury can be diagnosed, such as resulting from arthritis, arthropathy, hypertrophy, injury, pain, facet adjacent segment disease, or inflammation of the facet joint. The diagnosis can be further specified or confirmed for a particular facet joint of the facet joint cluster by analysis of medical images (e.g., MRI images) of the facet joint cluster of the cervical spine. The treatment for the facet joint injury or issue may include, but not be limited to, denervation of the sensory nerves at the indicated facet joint, such as the medial branch of the dorsal ramus. Such denervation may be accomplished via minimally invasive percutaneous cauterization of the sensory nerves extending adjacent the facet joint. Alternatively, the TP may be associated with a specific facet joint along the cervical spine, rather than a cluster of facet joints along the cervical spine and the diagnosed confirmed in association with medical images of the facet joint(s) of the cervical spine.

Notwithstanding the above exemplary diagnoses and treatments, it is recognized that at 219 various other appropriate diagnoses and/or treatments may be presented by the diagnostic software 23.

In 221, the diagnostic software 23 stores in a datastore the first output data (predicted diagnosis) and optional second output data (confidence level) output from the expert system in 217 and the corresponding treatment data of 219.

In 223, one or more medical experts evaluate the first output data (predicted diagnosis) and optional second output data (confidence level) and the corresponding treatment data for confirmation of the diagnosis of chronic spinal pain of the patient and appropriate treatment. The evaluation of 223 can evaluate the first output data (predicted diagnosis) and optional second output data (confidence level) against one or more diagnostic images (such as MRI images) of the back or neck of the patient. The evaluation of 223 can be performed by a medical doctor treating the patient, by a medical expert on behalf of an insurance company that has an obligation to pay for treatment of the patient, or by some other medical provider or related entity.

In 225, the diagnostic software 23 checks on the status of the evaluation of 223 to determine if the predicted diagnosis of chronic spinal pain of the patient and corresponding treatment is confirmed as the diagnosis of chronic spinal pain of the patient and appropriate treatment. If so, the operations continue to 227. If not, the operations continue to 229.

In 227, the diagnostic software 23 uses the data representing the predicted diagnosis and corresponding treatment to provide a diagnosis of chronic spinal pain of the patient and recommended treatment for the diagnosis. The diagnosis and recommended treatment can be communicated to the patient, a medical doctor treating the patient, an insurance company that has an obligation to pay for treatment of the patient, or some other medical provider or related entity. Such communication can involve messaging (such as an email message or SMS message directed to the appropriate recipient) or presentation of a suitable webpage or interface to the appropriate recipient, including via secure transmission that meets HIPAA (Health Insurance Portability and Accountability Act) compliance. For the treatment of chronic pain associated with the lumbar spine of the patient, the recommended treatment can refer to one or more of the treatments of the vertebral joints or bones or vertebral discs and associated musculature of the lumbar spine of the patient as summarized above. For the treatment of chronic pain associated with the thoracic spine of the patient, the recommended treatment can refer to one or more treatments of the vertebral joints or bones or vertebral discs of the thoracic spine of the patient as summarized above. For the treatment of chronic pain associated with the cervical spine of the patient, the recommended treatment can refer to one or more treatments of the vertebral joints or bones or vertebral discs of the cervical spine of the patient as summarized above.

In 229, the diagnostic software 23 triggers further medical evaluation of the patient. This trigger can be active for cases where the predicted diagnosis of chronic spinal pain is not confirmed, or where there is no predicted diagnosis of chronic spinal pain.

FIG. 3 illustrates the lower back of an example patient, with the skin of the lower back marked with a first set of visual indicia (lines AF1, AF2, AF3) corresponding to anatomical landmarks of the lower back of the patient. In this embodiment, the line AF1 extends down the center of lumbar spine of the patient, the line AF2 extends across bottom of lower back and crosses the tailbone/sacrum of the spine of the patient, and the line AF3 extends across midline of the lower back between the L2 and L3 vertebra of the lumbar spine of the patient.

FIG. 4 illustrates the lower back of an example patient together with circles and corresponding labels for vertebral joints or bones and vertebral discs of the lumbar spine that represent the locus of pain commonly experienced by patients together with a first set of visual indicia (lines AF1, AF2, AF3) corresponding to anatomical landmarks of the lower back of FIG. 3. The vertebral joints and vertebral discs and associated musculature of the lumbar spine of the patient as labeled in FIG. 4 can be referenced by the predicted diagnosis of the first output data of 217, which includes the following:
L1-L2 facet joint of the left side of the lumbar spine, labeled “L1-2FJ-L”;
L1-L2 facet joint of the right side of the lumbar spine, labeled “L1-2FJ-R”;
L2-L3 facet joint of the left side of the lumbar spine, labeled “L2-3FJ-L”;
L2-L3 facet joint of the right side of the lumbar spine, labeled “L2-3FJ-R”;
L3-L4 facet joint of the left side of the lumbar spine, labeled “L3-4FJ-L”;
L3-L4 facet joint of the right side of the lumbar spine, labeled “L3-4FJ-R”;
L4-L5 facet joint of the left side of the lumbar spine, labeled “L4-5FJ-L”;
L4-L5 facet joint of the right side of the lumbar spine, labeled “L4-5FJ-R”;
L5-S1 facet joint of the left side of the lumbar spine, labeled “L5-S1FJ-L”;
L5-S1 facet joint of the right side of the lumbar spine, labeled “L5-S1FJ-R”;
sacroiliac joint of the left side of the lumbar spine, labeled “SIJ-L”;
sacroiliac joint of the right side of the lumbar spine, labeled “SIJ-R”;
piriformis muscle adjacent the left side of the lumbar spine, labeled “PIR-L”;
piriformis muscle adjacent the right side of the lumbar spine, labeled “PIR-R”;
L1-L2 vertebral disc of the lumbar spine, labeled “L1-2D”;
L2-L3 vertebral disc of the lumbar spine, labeled “L2-3D”;
L3-L4 vertebral disc of the lumbar spine, labeled “L3-4D”;
L4-L5 vertebral disc of the lumbar spine, labeled “L4-5D”; and
L5-S1 vertebral disc of the lumbar spine, labeled “L5-S1D”.

FIG. 5A illustrates the lower back of an example patient that includes a first set of visual indicia (lines AF1, AF2, AF3) marked on the skin of the lower back that correspond to anatomical landmarks of the lower back of the patient together with a second set of visual indicia marked on the skin of the lower back of the patient, which includes a single pain point (PP) which is located at a locus of lower back pain experienced by the patient.

FIG. 5B illustrates the lower back of another example patient that includes a first set of visual indicia (lines AF1, AF2, AF3) marked on the skin of the lower back that correspond to anatomical landmarks of the lower back of the patient together with a second set of visual indicia marked on the skin of the lower back of the patient, which includes a single tender point (TP) which is located at a locus of lower back pain experienced by the patient.

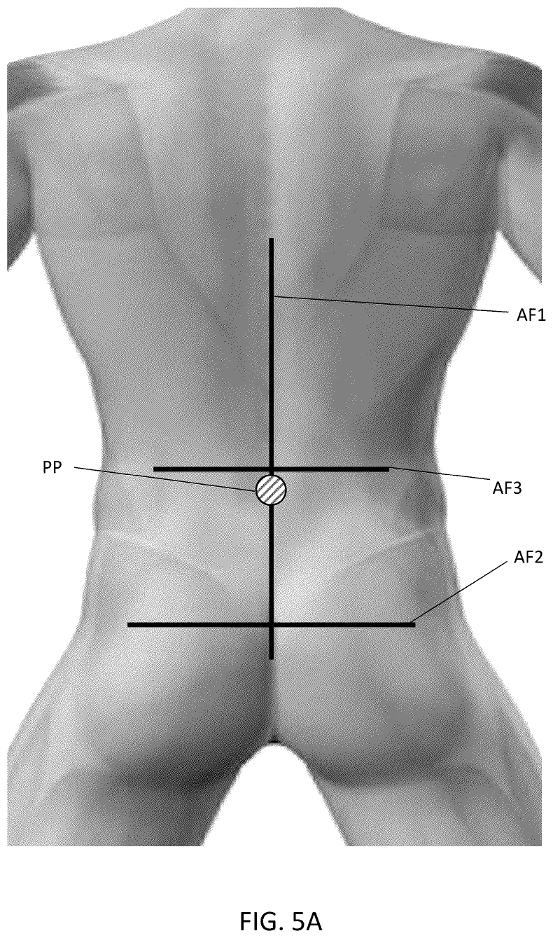

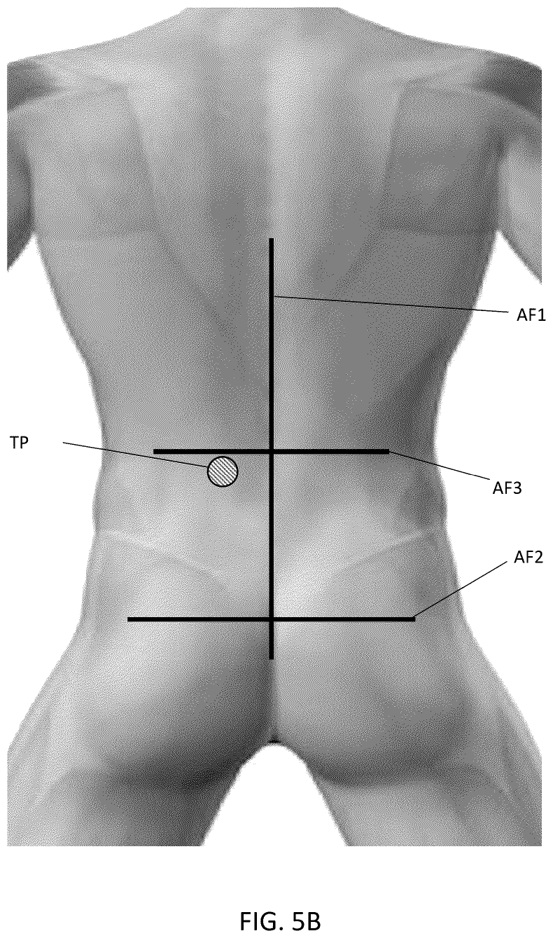

FIG. 6A illustrates example visual indicia and pain point feature data extracted from an image or infographic of a patient's back by the processing of 213 and 215 in the workflow of FIGS. 2A to 2C. In embodiments, the image processing of 213 can be configured to detect the lines AF1, AF2, AF3 (i.e., the first set of visual indica) in the image or infographic using suitable image processing techniques, such as a combination of edge detection (e.g., Canny edge detection as described in Canny, J., A Computational Approach To Edge Detection, IEEE Transactions on Pattern Analysis and Machine Intelligence, 8 (6): 679-698, 1986) and a Hough transform (e.g., see Duda, R. O.; Hart, P. E. (January 1972), “Use of the Hough Transformation to Detect Lines and Curves in Pictures,” Comm. ACM. 15:11-15. doi: 10.1145/361237.361242. S2CID 1105637). The pixels of the image or infographic can be rotated to align the detected line AF1 in a vertical orientation and the detected lines AF2, AF3 in a horizontal orientation. The image processing can determine the pixel coordinates (X_{AF1}, Y_{AF2}, Y_{AF3}) for the lines AF1, AF2, AF3 in the aligned image or infographic. The image processing can be configured to detect a circular or other-shaped marking of the pain point (i.e., the second set of visual indicia) in the aligned image or infographic using suitable image processing techniques, such as Hough transform or a convolution based technique. The image processing can determine the pixel coordinates (X_{PP}, Y_{PP}) of the center point of the detected circular or other-shaped marking in the aligned image or infographic. The pain point characteristic type (e.g., a pain point representing pain independent of applied pressure) can be determined by detecting the color, shading or other distinguishing property of the detected circular or other-shaped marking in the aligned image or infographic. Example tender point feature data can be calculated from the pixel coordinates of the lines AF1, AF2, AF3 and the pixel coordinates (X_{PP}, Y_{PP}) of the pain point (PP) as summarized in FIG. 6A.

FIG. 6B illustrates example visual indicia and tender point feature data extracted from an image or infographic of a patient's back by the processing of 213 and 215 in the workflow of FIGS. 2A to 2C. In embodiments, the image processing of 213 can be configured to detect the lines AF1, AF2, AF3 (i.e., the first set of visual indica) in the image or infographic using suitable image processing techniques, such as a combination of edge detection (e.g., Canny edge detection as described in Canny, J., A Computational Approach To Edge Detection, IEEE Transactions on Pattern Analysis and Machine Intelligence, 8 (6): 679-698, 1986) and a Hough transform (e.g., see Duda, R. O.; Hart, P. E. (January 1972), “Use of the Hough Transformation to Detect Lines and Curves in Pictures,” Comm. ACM. 15:11-15. doi: 10.1145/361237.361242. S2CID 1105637). The pixels of the image or infographic can be rotated to align the detected line AF1 in a vertical orientation and the detected lines AF2, AF3 in a horizontal orientation. The image processing can determine the pixel coordinates (X_{AF1}, Y_{AF2}, Y_{AF3}) for the lines AF1, AF2, AF3 in the aligned image or infographic. The image processing can be configured to detect a circular or other-shaped marking of the tender point (i.e., the second set of visual indicia) in the aligned image or infographic using suitable image processing techniques, such as Hough transform or a convolution based technique. The image processing can determine the pixel coordinates (X_{TP}, Y_{TP}) of the center point of the detected circular or other-shaped marking in the aligned image or infographic. The tender point characteristic type (e.g., a tender point representing pain that results from applied pressure) can be determined by detecting the color, shading or other distinguishing property of the detected circular or other-shaped marking in the aligned image or infographic. Example tender point feature data can be calculated from the pixel coordinates of the lines AF1, AF2, AF3 and the pixel coordinates (X_{TP}, Y_{TP}) of the tender point (TP) as summarized in FIG. 6B.

In embodiments, the workflow can processes the pixel coordinate data for the first and second sets of visual indicia and the pain characteristic data as extracted from the image or infographic and optionally one or more answers or responses to a questionnaire or diagnostic prompts that provides information that characterizes the pain experienced by the patient to generate feature data representing spatial location and associated pain characteristics of one or points (e.g., pain point(s) or tender point(s)) corresponding to locus of pain experienced by the patient.

For example, in one illustrative embodiment shown in FIG. 6A, the pain point feature data for a given pain point (PP) can include the following:
ratio of Y-offset of PP relative to AF3 to the Y-offset of the PP relative to AF2 (e.g., calculated as ABS ((Y_{PP}−Y_{AF2})/(Y_{AF3}−Y_{AF2}));
positive/negative property of Y-offset of PP relative to AF3 (e.g., where positive property of Y-offset of PP relative to AF3 is determined by Y_{PP}>Y_{AF3}, and the negative property of Y-offset of PP relative to AF3 is determined by Y_{PP}<Y_{AF3});
positive/negative property of Y-offset of PP relative to AF2 (e.g., where positive property of Y-offset of PP relative to AF2 is determined by Y_{PP}>Y_{AF2}, and the negative property of Y-offset of PP relative to AF2 is determined by Y_{PP}<Y_{AF2});
ratio of X-offset of PP relative to AF1 to the Y-offset of the AF3 relative to AF2 (e.g., calculated as ABS ((X_{PP}−Y_{AF2})/(Y_{AF3}−Y_{AF2}));
positive/negative property of X-offset of PP relative to AF1 (e.g., where positive property of X-offset of PP relative to AF1 is determined by X_{PP}>X_{AF1}, and the negative property of X-offset of PP relative to AF1 is determined by X_{PP}<X_{AF1});
Pain Characteristic Type for the PP.
   In embodiments, the spatial properties of the pain point represented by the pain point feature data can be invariant to zoom factor variability or other geometric distortions or deformations that can occur when the image or infographic is acquired at a non-ideal position or orientation relative to the back or neck of the patient. In other embodiments, the pain point feature data can include other spatial properties of the pain point (PP) calculated from the pixel coordinates of the lines AF1, AF2, AF3 and the pixel coordinates (X_{PP}, Y_{PP}) of the pain point (PP).

In another illustrative embodiment shown in FIG. 6B, the tender point feature data for a given tender point (TP) can include the following:
ratio of Y-offset of TP relative to AF3 to the Y-offset of the TP relative to AF2 (e.g., calculated as ABS ((Y_{TP}−Y_{AF2})/(Y_{AF3}−Y_{AF2}));
positive/negative property of Y-offset of TP relative to AF3 (e.g., where positive property of Y-offset of TP relative to AF3 is determined by Y_{TP}>Y_{AF3}, and the negative property of Y-offset of TP relative to AF3 is determined by Y_{TP}<Y_{AF3});
positive/negative property of Y-offset of TP relative to AF2 (e.g., where positive property of Y-offset of PP relative to AF2 is determined by Y_{TP}>Y_{AF2}, and the negative property of Y-offset of PP relative to AF2 is determined by Y_{TP}<Y_{AF2});
ratio of X-offset of TP relative to AF1 to the Y-offset of the AF3 relative to AF2 (e.g., calculated as ABS ((X_{TP}−Y_{AF2})/(Y_{AF3}−Y_{AF2}));
positive/negative property of X-offset of TP relative to AF1 (e.g., where positive property of X-offset of TP relative to AF1 is determined by X_{TP}>X_{AF1}, and the negative property of X-offset of TP relative to AF1 is determined by X_{TP}<X_{AF1});
Pain Characteristic Type for the TP.

In embodiments, the spatial properties of the tender point represented by the tender point feature data can be invariant to zoom factor variability or other geometric distortions or deformations that can occur when the image or infographic is acquired at a non-ideal position or orientation relative to the back or neck of the patient. In other embodiments, the tender point feature data can include other spatial properties of the tender point (TP) calculated from the pixel coordinates of the lines AF1, AF2, AF3 and the pixel coordinates (X_{TP}, Y_{TP}) of the tender point (TP).

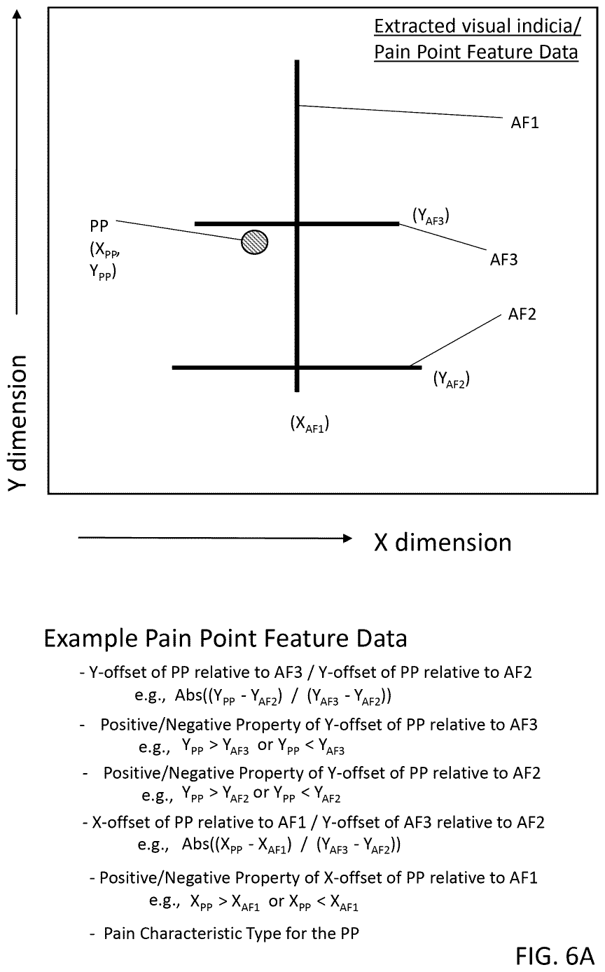

FIG. 7 illustrates example operations of the workflow of the present disclosure. In 701, patient-specific feature data representing spatial location and associated pain characteristic of one or more points corresponding to locus of pain experienced by the patient is generated from image processing.

In 703, the patient-specific feature data is formatted for input to an expert system 705. The expert system 705 can be a rule-based decision tree or artificial neural network or other machine learning system, which is configured to output first output data 707 representing a predicted diagnosis of chronic spinal pain of the patient as indicated by the feature data input and optionally second output data 709 representing a confidence level associated with the predicted diagnosis of the first output data 707. The rule-based decision tree can embody rules that link spatial location and pain characteristics of the pain points or tender points to corresponding predicted diagnosis and optional confidence level. For example, the rules can generate data characterizing spatial location for a set of anatomical features points of the user in the processed image. Such data can be based on offset of the anatomical landmarks (e.g., offset between AF2 and AF3) in the processed image as derived from the image processing.

For example, for diagnosing chronic pain of the lumbar spine of a patient, a set of anatomical feature points relating to the lumbar spine of the patient can include one or more of the following:
L1-L2 Disc
L2-L3 Disc
L3-L4 Disc
L4-L5 Disc
L5-S1 Disc
L1-L2 Facet Joint of the left side of the lumbar spine
L1-L2 Facet Joint of the right side of the lumbar spine
L2-L3 Facet Joint of the left side of the lumbar spine
L2-L3 Facet Joint of the right side of the lumbar spine
L3-L4 Facet Joint of the left side of the lumbar spine
L3-L4 Facet Joint of the right side of the lumbar spine
Left L4-L5 Facet Joint of the left side of the lumbar spine
L4-L5 Facet Joint of the right side of the lumbar spine
L5-S1 Facet Joint of the left side of the lumbar spine
L5-S1 Facet Joint of the right side of the lumbar spine
SI Joint of the left side of the lumbar spine
SI Joint of the right side of the lumbar spine
piriformis muscle adjacent the left side of the lumbar spine
piriformis muscle adjacent the right side of the lumbar spine
L1 Vertebral Body
L2 Vertebral Body
L3 Vertebral Body
L4 Vertebral Body
L5 Vertebral Body
Coccyx
   Some of these anatomical feature points are labeled by circles in FIG. 4.

In another example, for diagnosing chronic pain of the thoracic spine of a patient, a set of anatomical feature points relating to the thoracic spine of the patient can include one or more of the following:
first cluster of vertebral discs (which covers the T1-T2, T2-3, T3-T4 discs) of the thoracic spine;
second cluster of vertebral discs (which covers the T4-T5, T5-6, T6-T7 discs) of the thoracic spine;
third cluster of vertebral discs (which covers the T7-T8, T8-9, T9-T10 discs) of the thoracic spine;
fourth cluster of vertebral discs (which covers the T10-T11, T11-12, T12-L1 discs) of the thoracic spine;
first cluster of facet joints (which covers the facet joints located between T1 to T4) of the left side of the thoracic spine;
first cluster of facet joints (which covers the facet joints located between T1 to T4) of the right side of the thoracic spine;
second cluster of facet joints (which covers the facet joints located between T4 to T7) of the left side of the thoracic spine;
second cluster of facet joints (which covers the facet joints located between T4 to T7) of the right side of the thoracic spine;
third cluster of facet joints (which covers the facet joints located between T7 to T10) of the left side of the thoracic spine;
third cluster of facet joints (which covers the facet joints located between T7 to T10) of the right side of the thoracic spine;
fourth cluster of facet joints (which covers the facet joints located between T10 to L1) of the left side of the thoracic spine; and
fourth cluster of facet joints (which covers the facet joints located between T10 to L1) of the right side of the thoracic spine.

Some of these anatomical feature points are labeled by circles in FIG. 9. The clusters for the vertebral discs and the facet joints of the thoracic spine can be arranged in other groupings extending fewer or more vertebrae. By way of example only, the cluster size may extend 2-4 vertebrae.

In still another example, for diagnosing chronic pain of the cervical spine of a patient, a set of anatomical feature points relating to the cervical spine of the patient can include one or more of the following:
first cluster of vertebral discs (which covers the C2-C3, C3-C4, C4-C5 discs) of the cervical spine;
second cluster of vertebral discs (which covers the C5-C6, C6-C7, C7-T1 discs) of the cervical spine;
first cluster of facet joints (which covers the facet joints for C1 to C5) of the left side of the cervical spine;
first cluster of facet joints (which covers the facet joints for C1 to C5) of the right side of the cervical spine;
second cluster of facet joints (which covers the facet joints for C5 to T1) of the left side of the cervical spine; and
second cluster of facet joints (which covers the facet joints for C5 to T1) of the right side of the cervical spine

Some of these anatomical feature points are labeled by circles in FIG. 10. The clusters for the vertebral discs and the facet joints of the cervical spine can be arranged in other groupings extending fewer or more vertebrae. By way of example only, the cluster size may extend 2-4 vertebrae.

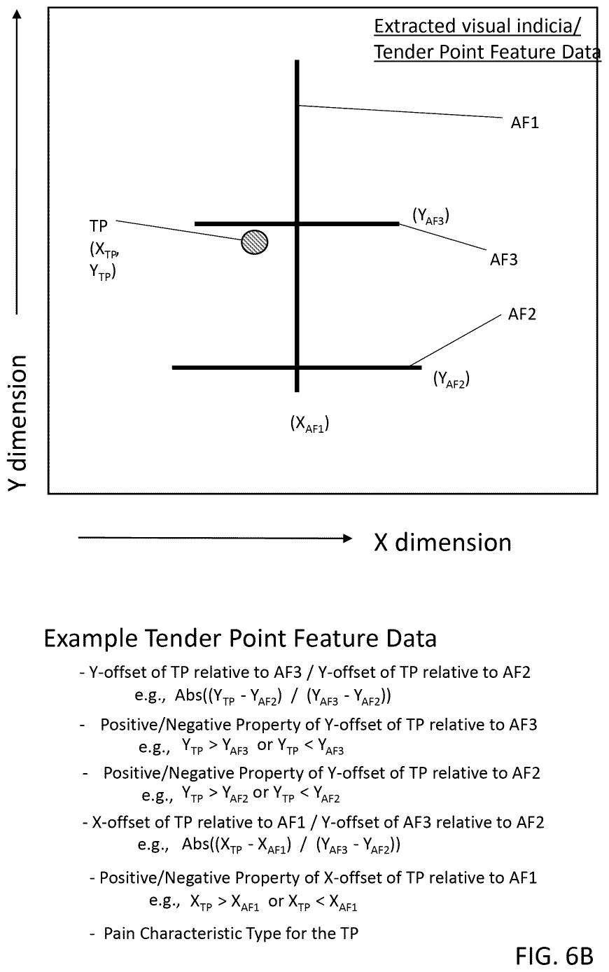

Data representing a predicted diagnosis of chronic spinal pain can be associated with a pain characteristic type (e.g., pain from applied pressure v. pain independent of applied pressure, and/or dull pain or sharp pain when pressure is applied) and given anatomical feature point in the set of anatomical features points. In this manner, different predicted diagnoses can be associated with the same locus of pain for the different pain characteristics. The rules can process the feature data (which characterizes the spatial location of the pain point or tender point in the processed image) and data characterizing spatial location for the set of the anatomical features points to identify the anatomical feature point that is closest to the location of the pain point or tender point in the processed image. The rules can then identify the data representing predicted diagnosis of chronic spinal pain that is associated with the pain characteristic type of the pain point and the identified “closest” anatomical feature point. The confidence level for the predicted diagnosis of spinal pain can be derived from the offset between the data characterizing spatial location of the pain point or tender point in the processed image (as characterized by the feature data) and the data characterizing spatial location of the identified “closest” anatomical feature point in the processed image. Other rule-based methods can be used to link spatial location and pain characteristics of the pain points or tender points to corresponding predicted diagnosis and optional confidence level. An artificial neural network or other machine learning system can be trained by training data to provide a similar answer product. An example of the training is described below with respect to FIG. 8.

FIG. 8 illustrates example operations for constructing a machine-learning system, which can be used as the expert system 705 in FIG. 7.

In 801, training data is obtained for a set of patients. The training data can include feature data representing location of chronic spinal pain experienced by each patient of the set (from data analysis or image processing) and associated labels representing diagnosis of chronic spinal pain and associated confidence level.

In 803, feature data input with associated labels is extracted from the training data.

In 805, the extracted feature data input with associated labels is used to train the machine-learning system.

In 807 and 809, the operations of the 803 and 805 are repeated for additional training data until the training ends. The amount of training data and number of iterations of the training can depend on the model complexity, number of features, and error tolerance. While no fixed rules exist, a popular guideline is to use 10 times or more training data examples relative to the features of the feature data input.

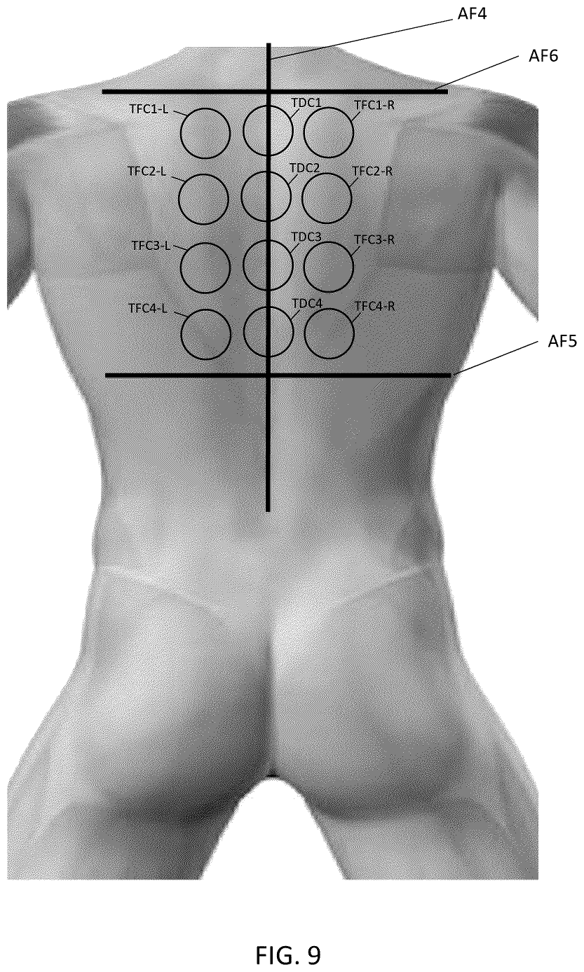

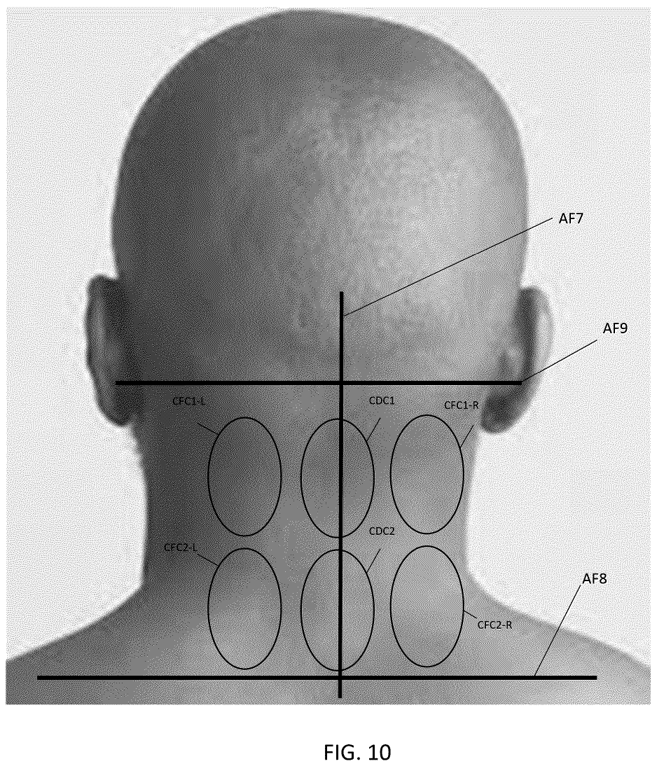

In another aspect, a kit can be provided to mark the back or neck of the patient with the first set of visual indicia corresponding to anatomical landmarks of the patient's back or neck and the second set of visual indica corresponding to locus of pain experienced by the patient. The kit can include at least one applique or decal 1101, which can be packaged in the form of a roll as shown in FIGS. 11A and 11B. The applique or decal 1101 can be configured to mark the back or neck of the patient with the first set of visual indicia corresponding to anatomical landmarks of the patient's back or neck as described herein. The applique or decal 1101 is formed by a flexible substrate having a first surface 1103 coated with a skin-adhering adhesive and a second surface 1105 disposed opposite the first surface 1103. A peel-away protective film 1107 can cover the first surface 1107 coated with the skin-adhering adhesive. In embodiments, the flexible substrate and the protective film 1107 of the applique or decal 1101 have a width in the range of 0.25 inches to 2 inches and a length in the range of 0.5 feet to 2.5 feet. The first surface 1103 can be adhered to the patient's skin covering an anatomical landmark of the patient's back or neck. In this configuration, the second surface 1105 provides visual indicia for marking the underlying anatomical landmark. The flexible substrate of the applique or decal 1101 can be formed from a web of paper, fabric, plastic or other flexible material. The visual indicia on the second surface 1105 can employ color, patterns or other visual properties to mark one or more anatomical landmarks of the patient's back or neck. Multiple appliques or decals 1101 can be provided (or cut from a roll by the user) and adhered to the patient's skin covering the anatomical landmarks of the patient's back or neck as described herein. In such configurations, the respective second surfaces 1105 of the appliques or decals provides visual indicia for marking the underlying anatomical landmarks of the patient's back or neck.

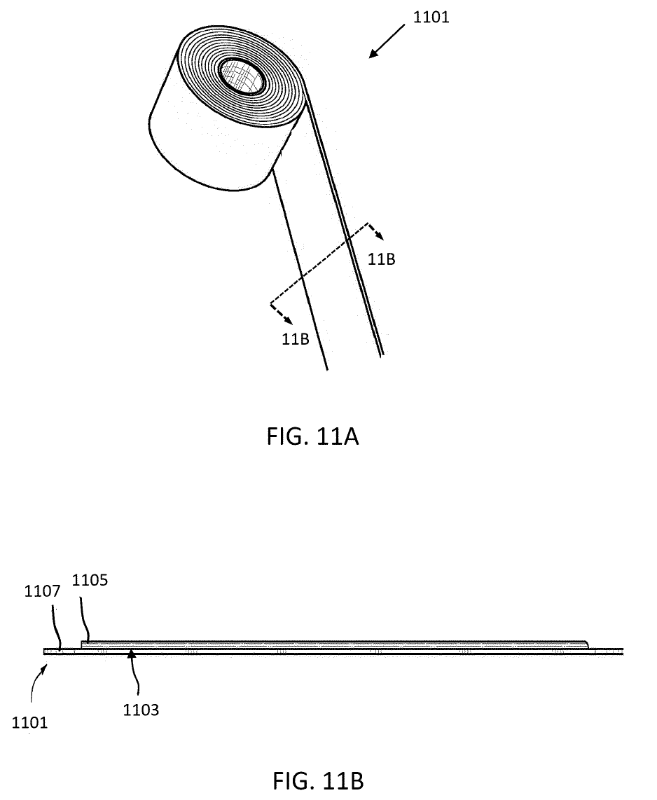

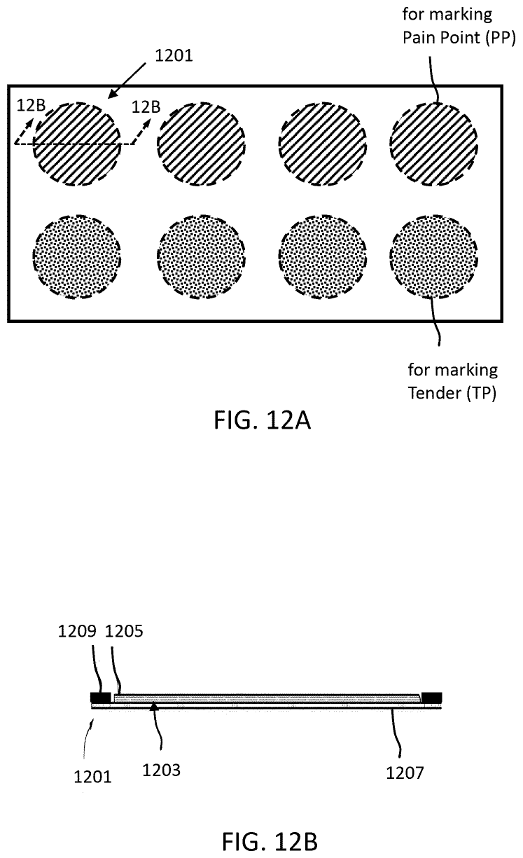

The kit can further include at least one applique or decal 1201, which can be packaged in the form of a sheet as shown in FIGS. 12A and 12B. The at least one applique or decal 1201 is configured to mark the back or neck of the patient with the second set of visual indicia corresponding to locus of pain experienced by the patient. Each applique or decal 1201 is formed by a flexible substrate having a first surface 1203 coated with a skin-adhering adhesive and a second surface 1205 disposed opposite the first surface 1203. A peel-away protective film 1207 can cover the first surface 1203 coated with the skin-adhering adhesive for the respective appliques or decals 1201. A flexible backing 1209 can be disposed opposite the film 1207 between the respective appliques or decals 1201. In embodiments, the outline of the flexible substrate and the protective film 1207 of the applique or decal 1201 can have a circular shape as best shown in FIG. 12A. In embodiments, the flexible substrate and the protective film 1207 of the applique or decal 1101 have a maximal dimension (i.e., diameter for circular shaped markers) in the range of 0.25 inches to 1 inch. The first surface 1203 can be adhered to the patient's skin covering a locus of pain experienced by the patient. The flexible substrate of the respective appliques or decals 1201 can be formed from a web of paper, fabric, plastic or other flexible material. The visual indicia on the second surface 1205 of the respective appliques or decals 1201 can employ color, patterns or other visual properties to mark the locus of pain experienced by the patient as well as the different characteristics of the pain. For example, the appliques or decals 1201 of FIGS. 12A and 12B employ two different colors or visual patterns to mark a Pain Point (PP) and Tender Point (PT) experienced by the patient as described herein. Multiple appliques or decals 1201 can be provided and adhered to the patient's skin covering the locus of pain experienced by the patient as described herein. In such configurations, the respective second surfaces of the appliques or decals 1201 provides visual indicia for marking the underlying loci of pain experienced by the patient.

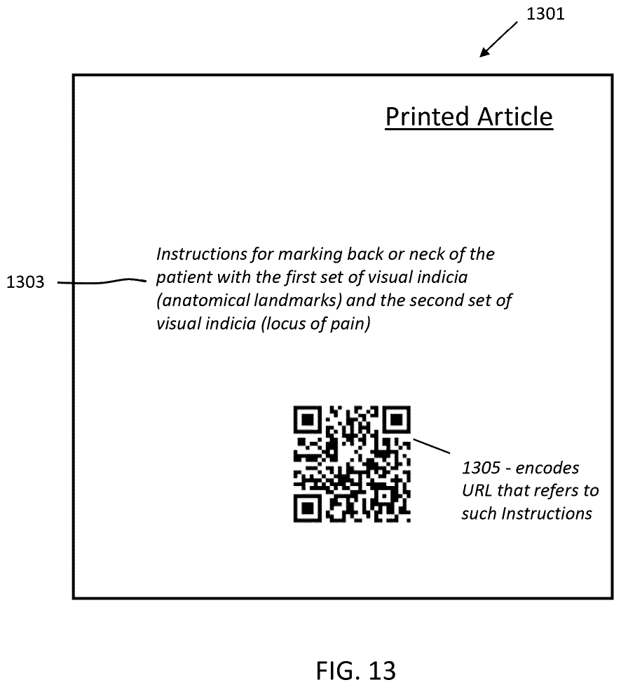

The kit can further include a printed article of manufacture that embodies or refers to written instructions for marking the back or neck of the patient with i) the first set of visual indicia (corresponding to the anatomical landmarks) provided by the at least one applique or decal 1101 of the kit and ii) the second set of visual indicia (corresponding to the locus of pain) provided by at least one applique or decal 1201 of the kit. An example printed article of manufacture (i.e., a printed sheet) 1301 is shown in FIG. 13, which can include a first part 1303 that includes written instructions for marking the back or neck of the patient with i) the first set of visual indicia (corresponding to the anatomical landmarks) provided by the at least one applique or decal 1101 and ii) the second set of visual indicia (corresponding to the locus of pain) provided by at least one applique or decal 1201. The printed article of manufacture 1301 can also include a second part 1303 (such as a bar code or QR code or other reference) that encodes a URL that references written instructions for marking the back or neck of the patient with i) the first set of visual indicia (corresponding to the anatomical landmarks) provided by the at least one applique or decal 1101 of the kit and ii) the second set of visual indicia (corresponding to the locus of pain) provided by at least one applique or decal 1201 of the kit.

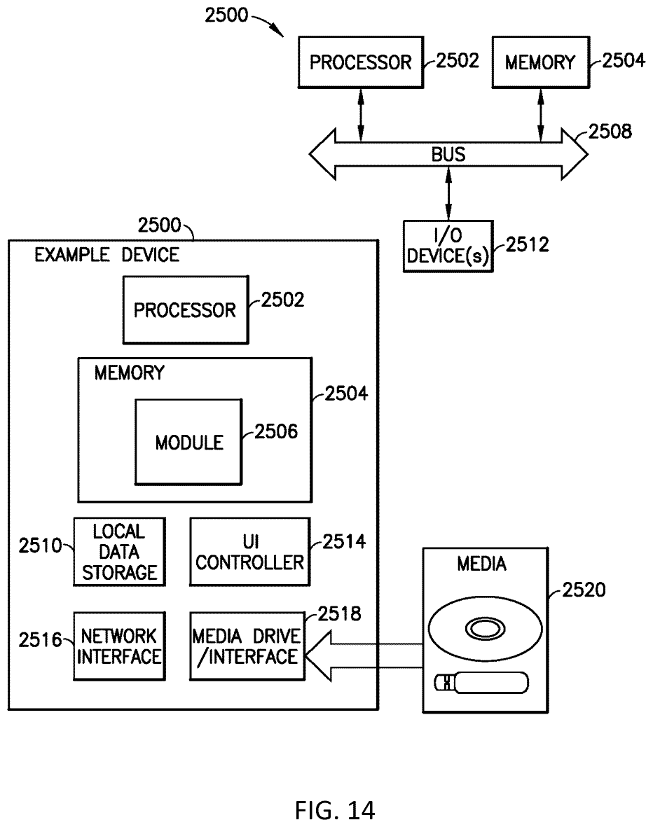

FIG. 14 illustrates an example device 2500, with a processor 2502 and memory 2504 that can be configured to implement various embodiments of the methods and processes as discussed in the present application, including some or all functions of the application server and diagnostic software as described herein. Memory 2504 can also host one or more databases and can include one or more forms of volatile data storage media such as random-access memory (RAM), and/or one or more forms of nonvolatile storage media (such as read-only memory (ROM), flash memory, and so forth).

Device 2500 is one example of a computing device or programmable device and is not intended to suggest any limitation as to scope of use or functionality of device 2500 and/or its possible architectures. For example, device 2500 can comprise one or more computing devices, programmable logic controllers (PLCs), etc.

Further, device 2500 should not be interpreted as having any dependency relating to one or a combination of components illustrated in device 2500. For example, device 2500 may include one or more computers, such as a laptop computer, a desktop computer, a mainframe computer, etc., or any combination or accumulation thereof.

Device 2500 can also include a bus 2508 configured to allow various components and devices, such as processors 2502, memory 2504, and local data storage 2510, among other components, to communicate with each other.

Bus 2508 can include one or more of any of several types of bus structures, including a memory bus or memory controller, a peripheral bus, an accelerated graphics port, and a processor or local bus using any of a variety of bus architectures. Bus 2508 can also include wired and/or wireless buses.

Local data storage 2510 can include fixed media (e.g., RAM, ROM, a fixed hard drive, etc.) as well as removable media (e.g., a flash memory drive, a removable hard drive, optical disks, magnetic disks, and so forth). One or more input/output (I/O) device(s) 2512 may also communicate via a user interface (UI) controller 2514, which may connect with I/O device(s) 2512 either directly or through bus 2508.

In one possible implementation, a network interface 2516 may communicate outside of device 2500 via a connected network. A media drive/interface 2518 can accept removable tangible media 2520, such as flash drives, optical disks, removable hard drives, software products, etc. In one possible implementation, logic, computing instructions, and/or software programs comprising elements of module 2506 may reside on removable media 2520 readable by media drive/interface 2518.

In one possible embodiment, input/output device(s) 2512 can allow a user (such as a human annotator) to enter commands and information into device 2500, and also allow information to be presented to the user and/or other components or devices. Examples of input device(s) 2512 include, for example, sensors, a keyboard, a cursor control device (e.g., a mouse), a microphone, a scanner, and any other input devices known in the art. Examples of output devices include a display device (e.g., a monitor or projector), speakers, a printer, a network card, and so on.

Various systems and processes of present disclosure may be described herein in the general context of software or program modules, or the techniques and modules may be implemented in pure computing hardware. Software generally includes routines, programs, objects, components, data structures, and so forth that perform particular tasks or implement particular abstract data types. An implementation of these modules and techniques may be stored on or transmitted across some form of tangible computer-readable media. Computer-readable media can be any available data storage medium or media that is tangible and can be accessed by a computing device. Computer readable media may thus comprise computer storage media. “Computer storage media” designates tangible media, and includes volatile and non-volatile, removable, and non-removable tangible media implemented for storage of information such as computer readable instructions, data structures, program modules, or other data. Computer storage media include, but are not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other tangible medium which can be used to store the desired information, and which can be accessed by a computer. Some of the methods and processes described above can be performed by a processor. The term “processor” should not be construed to limit the embodiments disclosed herein to any particular device type or system. The processor may include a computer system. The computer system may also include a computer processor (e.g., a microprocessor, microcontroller, digital signal processor, general-purpose computer, special-purpose machine, virtual machine, software container, or appliance) for executing any of the methods and processes described above.

The computer system may further include a memory such as a semiconductor memory device (e.g., a RAM, ROM, PROM, EEPROM, or Flash-Programmable RAM), a magnetic memory device (e.g., a diskette or fixed disk), an optical memory device (e.g., a CD-ROM), a PC card (e.g., PCMCIA card), or other memory device.

Alternatively or additionally, the processor may include discrete electronic components coupled to a printed circuit board, integrated circuitry (e.g., Application Specific Integrated Circuits (ASIC)), and/or programmable logic devices (e.g., a Field Programmable Gate Arrays (FPGA)). Any of the methods and processes described above can be implemented using such logic devices.

Some of the methods and processes described above can be implemented as computer program logic for use with the computer processor. The computer program logic may be embodied in various forms, including a source code form or a computer executable form. Source code may include a series of computer program instructions in a variety of programming languages (e.g., an object code, an assembly language, or a high-level language such as C, C++, or JAVA). Such computer instructions can be stored in a non-transitory computer readable medium (e.g., memory) and executed by the computer processor. The computer instructions may be distributed in any form as a removable storage medium with accompanying printed or electronic documentation (e.g., shrink wrapped software), preloaded with a computer system (e.g., on system ROM or fixed disk), or distributed from a server or electronic bulletin board over a communication system (e.g., the Internet or World Wide Web)

The systems, methods and processes discussed in the present application can provide the following advantages:
the diagnosis of chronic spinal pain and corresponding treatment is based on one or more images or infographics of the patient's back or neck, which can be generated at a convenient location for the patient without the patient traveling to and visiting a doctor's office or medical facility; the one or more images or infographics can be communicated to a remote system for diagnostic processing;
the diagnosis of chronic spinal pain and corresponding treatment is identified through the use of an expert system, which provides immediate access to knowledge and advice to patients and/or physicians and/or other medical provider entities;
the accuracy of the diagnosis of chronic spinal pain and corresponding treatment provided by the expert system can be verified (and possibly improve over time with ML training), thus avoiding common misdiagnosis and mistreatment of chronic spinal pain.

Although only a few example embodiments have been described in detail above, those skilled in the art will readily appreciate that many modifications are possible in the example embodiments without materially departing from this invention.

Accordingly, all such modifications are intended to be included within the scope of this disclosure as defined in the following claims. In the claims, means-plus-function clauses are intended to cover the structures described herein as performing the recited function and not only structural equivalents, but also equivalent structures.

What is claimed is:

1. A computer-implemented method of diagnosing chronic pain at a back area of patient, the back area including a spine and buttocks, the method comprising:
receiving and storing at least one image or infographic of the patient's back or neck, wherein the least one image or infographic includes a first set of visual indicia corresponding to anatomical landmarks of the patient's back area as well as a second set of visual indica corresponding to locus of pain experienced by the patient;
processing the at least one image or infographic to generate feature data representing spatial location and associated pain characteristics of one or more points corresponding to locus of pain experienced by the patient; and
inputting the feature data to an expert system that generates first output data representing a predicted diagnosis of chronic spinal pain of the patient as indicated by the feature data input.

2. The method according to claim 1, further comprising:
accessing a lookup table or database to determine treatment data representing a treatment corresponding to the first output data.

3. The method according to claim 2, further comprising:
communicating with a medical expert to evaluate at least the first output data and the treatment data for confirmation of the diagnosis of chronic spinal pain of the patient and appropriate treatment.

4. The method according to claim 3, wherein:
the evaluation involves evaluation of anatomical images of at least a portion of the patient's back area.

5. The method according to claim 3, further comprising:
when the predicted diagnosis and corresponding treatment are confirmed, use the first output data and the treatment data to provide a diagnosis of chronic pain at the back area of the patient and recommended treatment for the diagnosis.

6. The method according to claim 1, wherein:
the expert system further generates second output data representing a confidence level associated with the predicted diagnosis of chronic pain at the back area of the patient as indicated by the feature data input.

7. The method according to claim 6, further comprising:
accessing a lookup table or database to determine treatment data representing a treatment corresponding to the first output data and the second output data.

8. The method according to claim 7, further comprising:
communicating with a medical expert to evaluate at least the first output data, the second output data and the treatment data for confirmation of the diagnosis of chronic pain at the back area of the patient and appropriate treatment.

9. The method according to claim 8, further comprising:
when the predicted diagnosis and corresponding treatment are confirmed, use the first output data, the second output data and the treatment data to provide a diagnosis of chronic pain at the back area of the patient and recommended treatment for the diagnosis.

10. The method according to claim 1, wherein:
at least part of the operations of the method are performed by a cloud computing system, and the at least one image or infographic is generated by a user device remote from the cloud computing system and communicated to the cloud computing system from the user device through data communication over the Internet.

11. The method according to claim 1, wherein:
the least one image or infographic depicts the patient's lower back; and
the first set of visual indicia includes a line down center of the lumbar spine of the patient, a line across the bottom of lower back that crosses a tailbone/sacrum of the spine of the patient, and a line across a midline of the lower back of the patient between the L2 and L3 vertebrae of a lumbar spine of the patient.

12. The method according to claim 1, wherein:
the predicted diagnosis of chronic pain at the back area of the patient refers diagnoses a problem with one or more vertebral joints or bones or vertebral discs or associated musculature of the lumbar spine of the patient.

13. The method according to claim 12, wherein:
the predicted diagnosis of chronic pain at the back area of the patient refers to a specific facet joint at the lumber spine on the left or right side of the lumbar spine, a specific cervical disc at the lumber spine, or a left-side or right-side sacroiliac joint.

14. The method according to claim 1, wherein:
the predicted diagnosis of chronic pain at the back area of the patient refers to at least one of the following:
a piriformis muscle at a the left side of the lumbar spine, and
a piriformis muscle at a the right side of the lumbar spine.

15. The method according to claim 1, wherein:
the least one image or infographic depicts the patient's upper back; and
the first set of visual indicia includes a line down the center of the thoracic spine of the patient, a line across the bottom of the thoracic spine that is aligned with the bottom of the rib cage of the patient, and a line across the top of the thoracic spine that is aligned with the top of the opposed shoulder blades of the patient.

16. The method according to claim 1, wherein:
the predicted diagnosis of chronic pain of the patient refers to one or more vertebral joints or bones or vertebral discs of the thoracic spine of the patient.

17. The method according to claim 16, wherein:
the predicted diagnosis of chronic pain of the patient is directed to one of a plurality of clusters of facets joints on the right side of the thoracic spine, one of a plurality of clusters of facets joints on the left side of the thoracic spine, one of a plurality of a cluster of vertebral discs located within the thoracic spine.

18. The method according to claim 17, wherein:
the clusters of facet joints include groupings of 2-4 facet joints, and the clusters of vertebral discs include groupings of 2-4 vertebral discs.

19. The method according to claim 1, wherein:
the least one image or infographic depicts the patient's neck; and
the first set of visual indicia includes a line down the center of the cervical spine of the patient, a line across the bottom of the cervical spine that is aligned with the top of the opposed shoulder blades of the patient, and a line across the top of the cervical spine that is aligned with the base of the skull of the patient.

20. The method according to claim 1, wherein:
the predicted diagnosis of chronic spinal pain of the patient refers to one or more vertebral joints or bones or vertebral discs of the cervical spine of the patient.

21. The method according to claim 1, wherein:
the predicted diagnosis of chronic pain of the patient is directed to one of a plurality of clusters of facets joints on the right side of the cervical spine, one of a plurality of clusters of facets joints on the left side of the cervical spine, one of a plurality of a cluster of vertebral discs located within the cervical spine.

22. The method according to claim 1, wherein:
the first set of visual indicia are marked on the back of patient using marker(s), pen(s), tape, or stickers; and/or
the first set of visual indicia are marked on the back or neck of the patient by the patient or family member or friend or other person at home or at other premises remote from a doctor's office or treatment center.

23. The method according to claim 1, wherein:
the second set of visual indicia include a circular or other shaped marking at one or more locations corresponding to locus of pain experienced by the patient; and/or
distinction between the visual indicia of the first and second sets use varying color or patterns or other visual properties; and/or
the visual indicia of the second set correspond to different pain characteristics; and/or
distinction between the visual indicia of the different pain characteristics of the second set use varying color or patterns or other visual properties; and/or
the second set of visual indicia are marked on the back of the patient using marker(s) or pen(s) or stickers; and/or
the second set of visual indicia are marked on the back or neck of the patient by the patient or family member or friend or other person at home or at other premises remote from the doctor's office or treatment center.

24. The method according to claim 1, wherein:
the visual indicia of the second set distinguish pain from applied pressure from pain independent of applied pressure, and/or
the visual indicia of the second set distinguish pain dull pain or sharp pain when pressure is applied.

25. The method according to claim 1, wherein:
the expert system comprises a machine learning system trained from training data comprising feature data representing location of chronic spinal pain experienced by a patient associated label representing diagnosis of chronic spinal pain and optionally an associated confidence level.
